(19) United States
(12) Patent Application Publication
(10) Pub. No.: US 2008/0255007 A1
(43) Pub. Date: Oct. 16, 2008

(54) Substrate Material For Handling and Analyzing Samples

(75) Inventors: Dirk Jan Broer, Geldrop (NL); Roel Penterman, Eindhoven (NL); Emiel Peeters, Eindhoven (NL); Ralph Kurt, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/064,670

(22) PCT Filed: Aug. 17, 2006
(86) PCT No.: PCT/IB2006/052843

§ 371 Date: Feb. 25, 2008

(30) Foreign Application Priority Data
Aug. 26, 2005 (EP) 05107854.1

(51) Int. Cl. C40B 60/12; G01N 21/01; F15B 21/00; G01N 33/48

(52) U.S. Cl. 506/39; 422/82.05; 137/807

(57) ABSTRACT

The invention relates to a substrate material for analyzing one or more fluid samples for the presence, amount or identity of one or more analytes in the samples, whereby the substrate material is adapted in that way that a flow of the sample or parts thereof in and/or with the substrate material is influenced and/or caused by phase transitions, preferably photoinducable phase transitions, in selected areas of the substrate material.

The present invention is directed to the field of devices for the handling and/or detection of one or more analytes in a sample, especially to the field of devices for handling and the detection of biomolecules in solution.

The present invention is directed to the handling and the detection of analytes in fluids, especially to the detection of biomolecules in solution. The detection usually occurs in that way, that the fluid to be analyzed is provided on a substrate material, which contains binding substances for the analytes which are subject of the detection. Such a capture probe may be a corresponding DNA-strand in case the analyte is also a DNA-Strand. The analytes in the fluid, which are usually equipped with a label, preferably an optical fluorescence label, will then be captured by the binding substance (in case of two complementary DNA strands this process is called hybridization) and remain there even after the fluid is removed. The analyte may then be detected.

However, usually the fluid is simply brought on the sample without any possibility to control the flow inside the substrate material. Such a flow may be controlled by microfluidic technology, but this requires a sophisticated layout of the analysis device and cannot be applied in all applications.

It is therefore an object of the present invention to provide a device and/or a substrate material for a device, which allows a quicker detection with a minute amount of analyte that needs to be present in the fluid.

This object is solved by a substrate material according to claim 1 of the present invention. Accordingly, a substrate material for analyzing a sample for at least one analyte is provided, whereby a lighting means is arranged for causing photo-inducible phase transitions of a liquid crystal in the sample exerting a force to the sample thereby causing a flow of the sample. Further, this object is solved by a method according to claim 5, a device according to claim 6, and a system according to claim 8.

By doing so, a lesser amount of sample is needed and a sophisticated microfluidic layout of the device can be avoided.

The term “sample” according to the present invention includes fluid samples as well as solid samples which dissolve when being provided with the substrate material.

In the sense of the present invention, the term “flow of fluid or parts thereof in and/or with the substrate material” includes especially one or more of the following features:
According to one preferred embodiment of the present invention, the flow of the whole fluid in and/or with the substrate material is influenced and/or caused by phase transitions in selected areas of the substrate material;
According to one preferred embodiment of the present invention, the flow of parts of the fluid in and/or with the substrate material is influenced and/or caused by phase transitions in selected areas of the substrate material, preferably of particles or larger biomolecules in the fluid, as will be described later on;
According to one preferred embodiment, the flow of the whole fluid in the substrate material is influenced and/or caused by phase transitions in selected areas of the substrate material; for this reason, the substrate material is e.g. porous or allows a certain solubility of the fluid within the substrate material, as will be described later on;
According to one preferred embodiment, the flow of the whole fluid with the substrate material is influenced and/or caused by phase transitions in selected areas of the substrate material; for this reason, the phase transition in the substrate material influences or controls also e.g. further layers or other means which are allocated or provided with the substrate material.

In the sense of the present invention, the term “phase transition” means especially the transition from an ordered state to a less ordered state or an inverse transition form an less ordered to an ordered state.

A—non limiting—example for a phase transition that is especially meant with the present invention is the transition from the crystalline state to the amorphous isotropic state. It is known that the solubility of species is much higher in the amorphous state of matter than in the crystalline state. Further—non limiting—examples for a phase transition that is especially meant with the present invention are also the phase transitions relating liquid crystalline materials such as the transition from the nematic state to the isotropic state, the transition from the smectic state to the isotropic state or to the nematic state, further smectic phase transitions, for instance from the smectic B state to the smectic A state, where also a decrease in order is involved, transitions in solubility might occur.

In the sense of the present invention, the term “selected areas” may also include the substrate material as a whole. However usually it is preferred that only a part of the substrate material is subjected to a phase transition in order to influence and/or cause a flow of fluid in and/or with the sample.

According to a preferred embodiment of the present invention, the substrate material is adapted in that way that a directed flow of fluid or parts thereof in and/or with the substrate material is influenced and/or caused by phase transitions in selected areas of the substrate material.

The term “directed” means especially that the direction, strength, lateral dispersion and/or distribution of the flow of fluid or parts thereof in and/or with the substrate material is influenced and/or caused by phase transitions in selected areas of the substrate material.

According to a preferred embodiment of the present invention, the phase transitions in the substrate material include reversible phase transitions.

By doing so, the flow in and/or with the substrate material may be controlled easier and more precise. Furthermore it is also possible to re-use the substrate material.

According to a preferred embodiment of the present invention, the substrate material is adapted in that way the interaction of the fluid to and/or in the substrate material changes when a phase transfer occurs.

By interaction is especially meant and/or included
the solubility of the fluid or parts of the fluid within the substrate material
the dispersibility of the fluid within the substrate material
the dispersibility of analyte particles within the substrate material. In this regard, it is taken reference to the following paper: ‘Drag on particles in a nematic suspension by a moving nematic-isotropic interface’ by J L West et al. Physical Review E, 2002, which is hereby incorporated by reference.
the adhesion of the fluid or analyte particles to the substrate material

The term “dispersibility” in the sense of the present invention means or includes especially the ability to disperse the fluid in the substrate material and/or the ability to mix or to blend other than by dissolving.

According to a preferred embodiment of the present invention, the substrate material is adapted in that way that solubility and/or dispersibility and/or adhesion of the fluid or parts of the fluid to and/or in the substrate material changes when a phase transfer occurs.

By doing so, a flow of fluid in and/or with the substrate material can be caused and/or influenced quite easily. Preferably, the phase transition changes the substrate material from a state, where the solubility and/or dispersibility and/or adhesion of the fluid to and/or in the substrate material is high to a state, where the solubility and/or dispersibility and/or adhesion of the fluid to and/or in the substrate material is low. Then the fluid will flow from the area(s) of the substrate material, where this phase transition had occurred to different areas, which still are in the initial state (or in the opposite/different phase).

According to a preferred embodiment of the present invention, the substrate material is adapted in that way that a flow of macroparticles in the fluid in and/or with the substrate material is influenced and/or caused by phase transitions in selected areas of the substrate material. The term “macroparticles” in the fluid means especially larger biomolecules such as DNA-strands, peptides, enzymes, antibodies, biomarkers, and proteins. By doing so, an analysis of these particles in the fluid can be achieved more easily and effectively.

According to a preferred embodiment of the present invention, the substrate material is adapted in that way that a flow of macroparticles in the fluid in and/or with the substrate material is influenced and/or caused by phase transitions in selected areas of the substrate material, whereby the macroparticles have an average diameter and/or average dimension of ≧1 nm and ≦50 μm. These ranges have proven in practice to be best suitable within the present invention. Preferably, the macroparticles have an average diameter of ≧2 nm and ≦5 μm, more preferred macroparticles ≧5 nm and ≦1 μm and most preferred ≧10 nm and ≦0.1 μm.

According to a preferred embodiment of the present invention, the substrate material is adapted in that way that a flow of macroparticles in the fluid in and/or with the substrate material is influenced and/or caused by a solubility transition of the macroparticles upon the phase transition. By doing so, this solubility transition can be one of the driving forces for transport of the macroparticles from one location, being the location that has the state of the lowest solubility, to another location where the solubility is higher.

According to a preferred embodiment of the present invention, the substrate material is adapted in that way that a flow of macroparticles in the fluid in and/or with the substrate material is influenced and/or caused by a change in dispersibility of macroparticles. It has been found that in some applications, especially crystalline and liquid-crystalline materials show a tendency to transport particles to their domain boundaries. In the case of crystals it is the lattice energy of the crystal that expels material that does not fit in the crystal lattice to its boundary. In the case of liquid crystals the elastic energy of the liquid crystal when its alignment is disturbed by the presence of the macroparticle is the driving force for this behavior of expulsion.

According to another preferred embodiment of the present invention, the substrate material is adapted in that way that a size-selective or size-dependent flow of macroparticles in the fluid in and/or with the substrate material is influenced and/or caused by phase transitions in selected areas of the substrate material.

According to a preferred embodiment of the present invention, the substrate material is adapted in that way that more than one phase transition is possible with the substrate material. By doing so, a more controlled and in most applications even selective flow of the sample or parts thereof in and/or with the substrate material is feasible. Such a substrate material may be e.g. a liquid crystal material (as will be described later on), in which a phase transition from a nematic to a smectic as well as a phase transition from a nematic to an isotropic state is possible. Some particles or components of the sample may be caused only to flow when the phase transition from nematic to isotropic occurs, whereas others may also be forced to flow by a phase transition from the nematic to the smectic state or vice versa. By using these differences, a more differentiated flow of different components of the fluid is possible, thus resulting in a device which is capable of a higher resolution and velocity in analyzing the fluid.

According to a preferred embodiment of the present invention, the substrate material forms a layer with a thickness of ≧0.1 μm and ≦100 μm, preferably between ≧0.5 μm and ≦20 μm and most preferred between ≧1 μm and ≦10 μm. This has shown to be suitable in practice.

According to a preferred embodiment of the present invention, the phase transitions in the substrate material include photo-inducible phase transitions. By doing so, an effective phase transition can be achieved without affecting the fluid or parts thereof, especially without adversely affecting the capture of analytes in the sample by binding substances.

According to a preferred embodiment of the present invention, the phase transitions in the substrate material include photo-inducable phase transitions which occur upon irradiation with a wavelength of ≧250 nm and ≦1500 nm, preferably ≧300 nm and ≦800 nm and most preferred ≧350 nm and ≦500 nm. This has shown to be in practice best suitable for the present invention.

According to a preferred embodiment of the present invention, the phase transitions in the substrate material include temperature-inducible phase transitions. This allows a better control and monitoring of the selected areas of the substrate material simply by using heating means, e.g. heating plates and/or cooling means, e.g. Peltier-elements, which can be addressed quite precisely.

According to a preferred embodiment of the present invention, the phase transitions in the substrate material include temperature-inducible phase transitions with a transition temperature between ≧0° C. and ≦150° C., preferably between ≧20° C. and ≦120° C., more preferably between ≧30° C. and ≦100° C., and most preferred between ≧40° C. and ≦55° C. It has been shown in practice that these temperatures are most suitable.

According to a preferred embodiment of the present invention, the phase transitions in the substrate material include temperature-inducible phase transitions and photo-inducible phase transitions. By doing so, the phase transitions may be induced by either lighting and/or heating the substrate material.

It should be noted that in some applications under irradiation in the presence of photo-active species often the polymorphism of the compounds remains the same but that only the phase transition temperatures shift to other values. It is obvious for those skilled in this field that by adjusting both temperature and photochemistry a better control over the phases and their transitions can be achieved.

According to a preferred embodiment of the present invention, the substrate material includes a material with comprises an azo group.

In the sense of the present invention, the term “a material with comprises an azo group” means that the following functional group

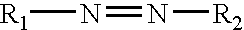

is present somewhere in the chemical structure of the material. R₁ and R₂ are independently selected from each other and may be identical or different.

Preferably, R₁ and/or R₂ are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

In case that the substrate material includes a material with comprises an azo group, a phase transfer may be reached by photochemically isomerization of the azo group.

Usually, an azo group exists in two conformations, cis and trans.

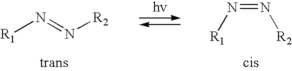

It is possible to isomerize between the two isomers photochemically and/or thermally. Usually the reaction from trans to cis will be conducted photochemically only, whereas the back reaction may be carried out thermally and photochemically.

Due to the different structure of the two isomers which have different isotropical as well as chemical properties, a shift in the azo-group containing material can be used to induce a phase transition in the substrate material. This goes especially for an insofar preferred embodiment (which will be described in great detail later on) in which the substrate material also contains a liquid crystal material.

According to a preferred embodiment of the present invention, the substrate material includes a material with comprises an azo group in that that one or more of the following materials and/or possibilities are present:
According to a preferred embodiment of the present invention, the substrate material includes a material which comprises an azo group and which is also capable to influence and/or cause a flow of fluid or parts thereof in and/or with the sample by undergoing phase transition(s).
According to a preferred embodiment of the present invention, the substrate material includes a material which comprises an azo group and which is—preferably upon isomerization—capable of influencing and/or causing a phase transition in or with a second material, which then influences and/or causes a flow of fluid or parts thereof in and/or with the sample.

According to a preferred embodiment of the present invention, the substrate material includes a material with comprises an azo group in that that one or more of the following materials and/or possibilities are present:
A material comprising an azo group and a further group capable of undergoing a phase transition in the same molecule; preferably, the material is a liquid crystal material;
A material comprising an azo group which is added to a second material capable of undergoing a phase transition in the same molecule; preferably, this second material is also a liquid crystal material;
A polymeric material where azo groups are present in side-chain moieties;
A polymeric liquid crystal material which also comprises azo groups; here the polymeric material may be a homopolymer and/or a copolymer.

According to a preferred embodiment of the present invention, the substrate material includes a material with comprises an azo group and which shows liquid crystal properties when the azo group is in one conformation and which shows different liquid crystal properties when the azo group is in the other conformation.

The term “different liquid crystal properties” includes one or more of the following:
the material has no or only very little liquid crystal properties when the azo group is in the other conformation;
the material still shows liquid crystal properties when the azo group is in the other conformation, but the phase transition points and/or conditions are different.

In order to illustrate the present invention in more detail, a few—merely illustrative and non-limiting—examples of a few molecules and/or substrate moieties with are usable within the present invention are now described.

According to a preferred embodiment of the present invention, an azo group can be the photo-reactive moiety in a molecule that forms a liquid crystalline phase. By irradiation the molecule can be converted from one isomeric form into another and visa versa, as is well known to those skilled in the field. The phase transition temperature of this molecule, e.g. the phase transition from the nematic phase to the isotropic phase, depends on the fact whether the azo group is in its E-shape (trans shape) or in its Z-shape (cis-shape).

When the E-isomer has a transition above and the Z-isomer is below the operating temperature, the material undergoes a phase transition. In convenient case, as an example, it will be a transition from the nematic phase to the isotropic phase. For instance the following azo molecules in their E-isomeric state have a liquid crystalline phase above their melting. The upper molecule melts at 86° C., then becomes smectic up to 98° C. above which it is nematic up to 110° C.

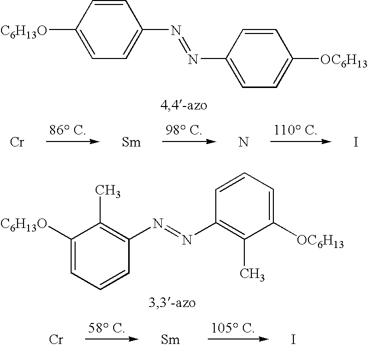

The lower molecule in it E-isomeric state melts at 58° C. and is smectic up to 105° C. Both materials do not show a liquid crystalline phase when converted into their Z-isomeric state. Some other examples of molecules showing a comparable behavior are:

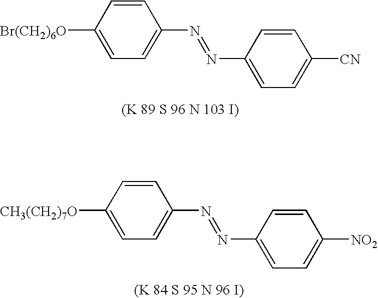

According to another preferred embodiment of the present invention, the azo moiety can also be incorporated in a molecule that is added as a guest dopant to a liquid crystalline host material such that the phase transitions, and also other properties such as solubility properties of analytes, are dominated by the host material. An example is given by a mixture based on a commercial blend of liquid crystal molecules E44 (Merck). This mixture has the following transition temperatures: K 6 N 100 I.

To this mixture various amounts of the liquid crystal azo molecule 3 are added. The phase transitions are measured and the samples are exposed to 366 nm light to convert the E isomer into the Z isomer (often not a complete conversion is obtained but the system reached the so-called photo-stationary state which under the given circumstances of light intensity, temperature, etc. gives a stationary conversion to the Z-isomer. Usually the concentration of Z-isomer is then around 90%). During exposure the nematic to isotropic temperature is measured again. The values are shown in the next table:

Nematic-to-isotropic transition
temperature (° C.)

| Conc. Azo molecule 3 in | Before 366 nm | During 366 nm |
|---|---|---|
| E44 (wt %) | exposure | exposure |
| 6 | 101 | 90 |
| 11 | 99 | 79 |
| 14 | 97 | 70 |

This means when the mixture containing the 14 wt % azo molecule 3 is operated at 80° C. it will undergo the phase transition from nematic to isotropic. For further details of this photo-induced transition reference can be made to: C H Legge and G R Mitchel, J. Phys. D: Appl. Phys. 25 (1992) 492-499, which is hereby incorporated by reference.

According to a further preferred embodiment of the present invention, rather than using low-molar-mass liquid crystals also polymeric liquid crystals and/or mixtures of polymeric liquid crystals and low-molar-mass liquid crystals can be used. For polymeric liquid crystals it is convenient to attach the azo containing group as a side group to a polymeric chain. Possibly also use can be made of a co-polymer where the side groups can be mixtures of groups that contain the azo group, groups that provide or modify liquid crystal behavior and groups that optimize the polymer on other properties such as solubility behavior of the analyte.

One merely exemplarily example is the following:

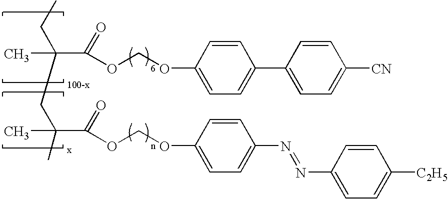

| | | | phase transition |
|---|---|---|---|
| polymer | x | n | temp. (° C.) |
| MACB-AB3 | 7 | 3 | G 51 N 114 I |
| MACB-AB6 | 6 | 6 | G 44 N 115 I |
| MACB-AB11 | 7 | 11 | G 36 N 113 I |
| AB6 | 100 | 6 | G 95 S 116 I |

According to a preferred embodiment of the present invention, the substrate material includes a material with comprises at least one group with the structure I:

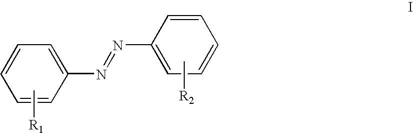

wherein R1 and/or R2 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

The term “includes a material with comprises at least one group with the structure I” in the sense of the present invention means especially that the chemical structural moiety according to structure I is present somewhere in the substrate material and/or in materials provided with the substrate material.

Substances like these have proven themselves to be best suitable in practice.

Generic group definition: Throughout the description and claims generic groups have been used, for example alkyl, alkoxy, aryl. Unless otherwise specified the following are preferred groups that may be applied to generic groups found within compounds disclosed herein:

alkyl: linear and branched C1-C8-alkyl,

long-chain alkyl: linear and branched C5-C20 alkyl

alkenyl: C2-C6-alkenyl,

cycloalkyl: C3-C8-cycloalkyl,

alkoxy: C1-C6-alkoxy,

long-chain alkoxy: linear and branched C5-C20 alkoxy

alkylene: selected from the group consisting of:

methylene; 1,1-ethylene; 1,2-ethylene; 1,1-propylidene; 1,2-propylene; 1,3-propylene; 2,2-propylidene; butan-2-ol-1,4-diyl; propan-2-ol-1,3-diyl; 1,4-butylene; cyclohexane-1,1-diyl; cyclohexan-1,2-diyl; cyclohexan-1,3-diyl; cyclohexan-1,4-diyl; cyclopentane-1,1-diyl; cyclopentan-1,2-diyl; and cyclopentan-1,3-diyl,

aryl: selected from homoaromatic compounds having a molecular weight under 300,

arylene: selected from the group consisting of: 1,2-phenylene; 1,3-phenylene; 1,4-phenylene; 1,2-naphtalenylene; 1,3-naphtalenylene; 1,4-naphtalenylene; 2,3-naphtalenylene; 1-hydroxy-2,3-phenylene; 1-hydroxy-2,4-phenylene; 1-hydroxy-2,5-phenylene; and 1-hydroxy-2,6-phenylene,

heteroaryl: selected from the group consisting of: pyridinyl; pyrimidinyl; pyrazinyl; triazolyl; pyridazinyl; 1,3,5-triazinyl; quinolinyl; isoquinolinyl; quinoxalinyl; imidazolyl; pyrazolyl; benzimidazolyl; thiazolyl; oxazolidinyl; pyrrolyl; carbazolyl; indolyl; and isoindolyl, wherein the heteroaryl may be connected to the compound via any atom in the ring of the selected heteroaryl,

heteroarylene: selected from the group consisting of: pyridindiyl; quinolindiyl; pyrazodiyl; pyrazoldiyl; triazolediyl; pyrazindiyl; and imidazolediyl, wherein the heteroarylene acts as a bridge in the compound via any atom in the ring of the selected heteroarylene, more specifically preferred are: pyridin-2,3-diyl; pyridin-2,4-diyl; pyridin-2,5-diyl; pyridin-2,6-diyl; pyridin-3,4-diyl; pyridin-3,5-diyl; quinolin-2,3-diyl; quinolin-2,4-diyl; quinolin-2,8-diyl; isoquinolin-1,3-diyl; isoquinolin-1,4-diyl; pyrazol-1,3-diyl; pyrazol-3,5-diyl; triazole-3,5-diyl; triazole-1,3-diyl; pyrazin-2,5-diyl; and imidazole-2,4-diyl, a —C1-C6-heterocycloalkyl, wherein the heterocycloalkyl of the —C1-C6-heterocycloalkyl is, selected from the group consisting of: piperidinyl; piperidine; 1,4-piperazine, tetrahydrothiophene; tetrahydrofuran; 1,4,7-triazacyclononane; 1,4,8,11-tetraazacyclotetradecane; 1,4,7,10,13-pentaazacyclopentadecane; 1,4-diaza-7-thia-cyclononane; 1,4-diaza-7-oxa-cyclononane; 1,4,7,10-tetraazacyclododecane; 1,4-dioxane; 1,4,7-trithia-cyclononane; pyrrolidine; and tetrahydropyran, wherein the heterocycloalkyl may be connected to the —C1-C6-alkyl via any atom in the ring of the selected heterocycloalkyl,

heterocycloalkylene: selected from the group consisting of: piperidin-1,2-ylene; piperidin-2,6-ylene; piperidin-4,4-ylidene; 1,4-piperazin-1,4-ylene; 1,4-piperazin-2,3-ylene; 1,4-piperazin-2,5-ylene; 1,4-piperazin-2,6-ylene; 1,4-piperazin-1,2-ylene; 1,4-piperazin-1,3-ylene; 1,4-piperazin-1,4-ylene; tetrahydrothiophen-2,5-ylene; tetrahydrothiophen-3,4-ylene; tetrahydrothiophen-2,3-ylene; tetrahydrofuran-2,5-ylene; tetrahydrofuran-3,4-ylene; tetrahydrofuran-2,3-ylene; pyrrolidin-2,5-ylene; pyrrolidin-3,4-ylene; pyrrolidin-2,3-ylene; pyrrolidin-1,2-ylene; pyrrolidin-1,3-ylene; pyrrolidin-2,2-ylidene; 1,4,7-triazacyclonon-1,4-ylene; 1,4,7-triazacyclonon-2,3-ylene; 1,4,7-triazacyclonon-2,9-ylene; 1,4,7-triazacyclonon-3,8-ylene; 1,4,7-triazacyclonon-2,2-ylidene; 1,4,8,11-tetraazacyclotetradec-1,4-ylene; 1,4,8,11-tetraazacyclotetradec-1,8-ylene; 1,4,8,11-tetraazacyclotetradec-2,3-ylene; 1,4,8,11-tetraazacyclotetradec-2,5-ylene; 1,4,8,11-tetraazacyclotetradec-1,2-ylene; 1,4,8,11-tetraazacyclotetradec-2,2-ylidene; 1,4,7,10-tetraazacyclododec-1,4-ylene; 1,4,7,10-tetraazacyclododec-1,7-ylene; 1,4,7,10-tetraazacyclododec-1,2-ylene; 1,4,7,10-tetraazacyclododec-2,3-ylene; 1,4,7,10-tetraazacyclododec-2,2-ylidene; 1,4,7,10,13 pentaazacyclopentadec-1,4-ylene; 1,4,7,10,13-pentaazacyclopentadec-1,7-ylene; 1,4,7,10,13-pentaazacyclopentadec-2,3-ylene; 1,4,7,10,13-pentaazacyclopentadec-1,2-ylene; 1,4,7,10,13-pentaazacyclopentadec-2,2-ylidene; 1,4-diaza-7-thia-cyclonon-1,4-ylene; 1,4-diaza-7-thia-cyclonon-1,2-ylene; 1,4-diaza-7thia-cyclonon-2,3-ylene; 1,4-diaza-7-thia-cyclonon-6,8-ylene; 1,4-diaza-7-thia-cyclonon-2,2-ylidene; 1,4-diaza-7-oxacyclonon-1,4-ylene; 1,4-diaza-7-oxa-cyclonon-1,2-ylene; 1,4-diaza-7-oxa-cyclonon-2,3-ylene; 1,4-diaza-7-oxa-cyclonon-6,8-ylene; 1,4-diaza-7-oxa-cyclonon-2,2-ylidene; 1,4-dioxan-2,3-ylene; 1,4-dioxan-2,6-ylene; 1,4-dioxan-2,2-ylidene; tetrahydropyran-2,3-ylene; tetrahydropyran-2,6-ylene; tetrahydropyran-2,5-ylene; tetrahydropyran-2,2-ylidene; 1,4,7-trithia-cyclonon-2,3-ylene; 1,4,7-trithia-cyclonon-2,9-ylene; and 1,4,7-trithia-cyclonon-2,2-ylidene,

heterocycloalkyl: selected from the group consisting of: pyrrolinyl; pyrrolidinyl; morpholinyl; piperidinyl; piperazinyl; hexamethylene imine; 1,4-piperazinyl; tetrahydrothiophenyl; tetrahydrofuranyl; 1,4,7-triazacyclononanyl; 1,4,8,11-tetraazacyclotetradecanyl; 1,4,7,10,13-pentaazacyclopentadecanyl; 1,4-diaza-7-thiacyclononanyl; 1,4-diaza-7-oxa-cyclononanyl; 1,4,7,10-tetraazacyclododecanyl; 1,4-dioxanyl; 1,4,7-trithiacyclononanyl; tetrahydropyranyl; and oxazolidinyl, wherein the heterocycloalkyl may be connected to the compound via any atom in the ring of the selected heterocycloalkyl,

amine: the group —N(R)₂ wherein each R is independently selected from: hydrogen; C1-C6-alkyl; C1-C6-alkyl-C6H5; and phenyl, wherein when both R are C1-C6-alkyl both R together may form an —NC3 to an —NC5 heterocyclic ring with any remaining alkyl chain forming an alkyl substituent to the heterocyclic ring,

halogen: selected from the group consisting of: F; Cl; Br and I,

pseudohalogen: selected from the group consisting of —CN, —SCN, —OCN, N3, —CNO, —SeCN

sulphonate: the group —S(O)₂OR, wherein R is selected from: hydrogen; C1-C6-alkyl; phenyl; C1-C6-alkyl-C6H5; Li; Na; K; Cs; Mg; and Ca,

sulphate: the group —OS(O)₂OR, wherein R is selected from: hydrogen; C1-C6-alkyl; phenyl; C1-C6-alkyl-C6H5; Li; Na; K; Cs; Mg; and Ca,

sulphone: the group —S(O)2R, wherein R is selected from: hydrogen; C1-C6-alkyl; phenyl; C1-C6-alkyl-C6H5 and amine (to give sulphonamide) selected from the group: —NR′2, wherein each R′ is independently selected from: hydrogen; C1-C6-alkyl; C1C6-alkyl-C6H5; and phenyl, wherein when both R′ are C1-C6-alkyl both R′ together may form an —NC3 to an —NCS heterocyclic ring with any remaining alkyl chain forming an alkyl substituent to the heterocyclic ring,

carboxylate derivative: the group —C(O)OR, wherein R is selected from: hydrogen; C1-C6-alkyl; phenyl; C1-C6-alkyl-C6H5; Li; Na; K; Cs; Mg; and Ca,

carbonyl derivative: the group —C(O)R, wherein R is selected from: hydrogen; C1-C6-alkyl; phenyl; C1-C6-alkyl-C6H5 and amine (to give amide) selected from the group: —NR′2, wherein each R′ is independently selected from: hydrogen; C1-C6-alkyl; C1-C6-alkyl-C6H5; and phenyl, wherein when both R′ are C1-C6-alkyl both R′ together may form an —NC3 to an —NC5 heterocyclic ring with any remaining alkyl chain forming an alkyl substituent to the heterocyclic ring,

phosphonate: the group —P(O) (OR)₂, wherein each R is independently selected from: hydrogen; C1-C6-alkyl; phenyl; C1-C6-alkyl-C6H5; Li; Na; K; Cs; Mg; and Ca,

phosphate: the group —OP(O)(OR)2, wherein each R is independently selected from: hydrogen; C1-C6-alkyl; phenyl; C1-C6-alkyl-C6H5; Li; Na; K; Cs; Mg; and Ca,

phosphine: the group —P(R)₂, wherein each R is independently selected from: hydrogen; C1-C6-alkyl; phenyl; and C1-C6-alkyl-C6H5,

phosphine oxide: the group —P(O)R2, wherein R is independently selected from: hydrogen; C1-C6-alkyl; phenyl; and C1-C6-alkyl-C6H5; and amine (to give phosphonamidate) selected from the group: —NR′2, wherein each R′ is independently selected from: hydrogen; C1-C6-alkyl; C1-C6-alkyl-C6H5; and phenyl, wherein when both R′ are C1-C6-alkyl both R′ together may form an —NC3 to an —NC5 heterocyclic ring with any remaining alkyl chain forming an alkyl substituent to the heterocyclic ring.

polyether: chosen from the group comprising-(O—CH₂—CH(R))ₙ—OH and —(O—CH₂—CH(R))ₙ—H whereby R is independently selected from: hydrogen, alkyl, aryl, halogen and n is from 1 to 250.

Unless otherwise specified the following are more preferred group restrictions that may be applied to groups found within compounds disclosed herein:

alkyl: linear and branched C1-C6-alkyl,

long-chain alkyl: linear and branched C5-C10 alkyl, preferably linear C6-C8 alkyl

alkenyl: C3-C6-alkenyl,

cycloalkyl: C6-C8-cycloalkyl,

alkoxy: C1-C4-alkoxy,

long-chain alkoxy: linear and branched C5-C10 alkoxy, preferably linear C6-C8 alkoxy,

alkylene: selected from the group consisting of: methylene; 1,2-ethylene; 1,3-propylene; butan-2-ol-1,4-diyl; 1,4-butylene; cyclohexane-1,1-diyl; cyclohexan-1,2-diyl; cyclohexan-1,4-diyl; cyclopentane-1,1-diyl; and cyclopentan-1,2-diyl,

aryl: selected from group consisting of: phenyl; biphenyl; naphthalenyl; anthracenyl; and phenanthrenyl,

arylene: selected from the group consisting of: 1,2-phenylene; 1,3-phenylene; 1,4-phenylene; 1,2-naphtalenylene; 1,4-naphtalenylene; 2,3-naphtalenylene and 1-hydroxy-2,6-phenylene,

heteroaryl: selected from the group consisting of:

pyridinyl; pyrimidinyl; quinolinyl; pyrazolyl; triazolyl; isoquinolinyl; imidazolyl; and oxazolidinyl, wherein the heteroaryl may be connected to the compound via any atom in the ring of the selected heteroaryl, heteroarylene: selected from the group consisting of: pyridin 2,3-diyl; pyridin-2,4-diyl; pyridin-2,6-diyl; pyridin-3,5-diyl; quinolin-2,3-diyl; quinolin-2,4-diyl; isoquinolin-1,3-diyl; isoquinolin-1,4-diyl; pyrazol-3,5-diyl; and imidazole-2,4-diyl,

heterocycloalkyl: selected from the group consisting of:

pyrrolidinyl; morpholinyl; piperidinyl; piperidinyl; 1,4-piperazinyl; tetrahydrofuranyl; 1,4,7-triazacyclononanyl; 1,4,8,11-tetraazacyclotetradecanyl; 1,4,7,10,13-pentaazacyclopentadecanyl; 1,4,7,10-tetraazacyclododecanyl; and piperazinyl, wherein the heterocycloalkyl may be connected to the compound via any atom in the ring of the selected heterocycloalkyl, heterocycloalkylene: selected from the group consisting of:

piperidin-2,6-ylene; piperidin-4,4-ylidene; 1,4-piperazin-1,4-ylene; 1,4-piperazin-2,3-ylene; 1,4-piperazin-2,6-ylene; tetrahydrothiophen-2,5-ylene; tetrahydrothiophen-3,4-ylene; tetrahydrofuran-2,5-ylene; tetrahydrofuran-3,4-ylene; pyrrolidin-2,5-ylene; pyrrolidin-2,2-ylidene; 1,4,7-triazacyclonon-1,4-ylene; 1,4,7-triazacyclonon-2,3-ylene; 1,4,7-triazacyclonon-2,2-ylidene; 1,4,8,11-tetraazacyclotetradec-1,4-ylene; 1,4,8,11-tetraazacyclotetradec-1,8-ylene; 1,4,8,11-tetraazacyclotetradec-2,3-ylene; 1,4,8,11-tetraazacyclotetradec-2,2-ylidene; 1,4,7,10-tetraazacyclododec-1,4-ylene; 1,4,7,10-tetraazacyclododec-1,7-ylene; 1,4,7,10-tetraazacyclododec-2,3-ylene; 1,4,7,10-tetraazacyclododec-2,2-ylidene; 1,4,7,10,13-pentaazacyclopentadec-1,4-ylene; 1,4,7,10,13-pentaazacyclopentadec-1,7-ylene; 1,4-diaza-7-thia-cyclonon-1,4-ylene; 1,4-diaza-7-thia-cyclonon-2,3-ylene; 1,4-diaza-7-thia cyclonon-2,2-ylidene; 1,4-diaza-7-oxa-cyclonon-1,4-ylene; 1,4-diaza-7-oxa-cyclonon-2,3-ylene; 1,4-diaza-7-oxa-cyclonon-2,2-ylidene; 1,4-dioxan-2,6-ylene; 1,4-dioxan-2,2-ylidene; tetrahydropyran-2,6-ylene; tetrahydropyran-2,5-ylene; and tetrahydropyran-2,2-ylidene, a —C1-C6-alkyl-heterocycloalkyl, wherein the heterocycloalkyl of the —C1-C6-heterocycloalkyl is selected from the group consisting of: piperidinyl; 1,4-piperazinyl; tetrahydrofuranyl; 1,4,7-triazacyclononanyl; 1,4,8,11-tetraazacyclotetradecanyl; 1,4,7,10,13-pentaazacyclopentadecanyl; 1,4,7,10-tetraazacyclododecanyl; and pyrrolidinyl, wherein the heterocycloalkyl may be connected to the —C1-C6-alkyl via any atom in the ring of the selected heterocycloalkyl,

amine: the group —N(R)2, wherein each R is independently selected from: hydrogen; C1-C6-alkyl; and benzyl,

halogen: selected from the group consisting of: F and Cl,

sulphonate: the group —S(O)2OR, wherein R is selected from: hydrogen; C1-C6-alkyl; Na; K; Mg; and Ca,

sulphate: the group —OS(O)2OR, wherein R is selected from: hydrogen; C1-C6-alkyl; Na; K; Mg; and Ca,

sulphone: the group —S(O)2R, wherein R is selected from: hydrogen; C1-C6-alkyl; benzyl and amine selected from the group: —NR′2, wherein each R′ is independently selected from:

hydrogen; C1-C6-alkyl; and benzyl,

carboxylate derivative: the group —C(O)OR, wherein R is selected from hydrogen; Na; K; Mg; Ca; C1-C6-alkyl; and benzyl,

carbonyl derivative: the group: —C(O)R, wherein R is selected from: hydrogen; C1-C6-alkyl; benzyl and amine selected from the group: —NR′2, wherein each R′ is independently selected from: hydrogen; C1-C6-alkyl; and benzyl,

phosphonate: the group —P(O)(OR)₂, wherein each R is independently selected from: hydrogen; C1-C6-alkyl; benzyl; Na; K; Mg; and Ca,

phosphate: the group —OP(O)(OR)2, wherein each R is independently selected from: hydrogen; C1-C6-alkyl; benzyl; Na; K; Mg; and Ca,

phosphine: the group —P(R)₂, wherein each R is independently selected from: hydrogen; C1-C6-alkyl; and benzyl,

phosphine oxide: the group —P(O)R2, wherein R is independently selected from: hydrogen; C1-C6-alkyl; benzyl and amine selected from the group: —NR′2, wherein each R′ is independently selected from: hydrogen; C1-C6-alkyl; and benzyl.

polyether: chosen from the group comprising-(O—CH₂—CH(R))ₙ—OH and —(O—CH₂—CH(R))ₙ—H whereby R is independently selected from: hydrogen, methyl, halogen and n is from 5 to 50, preferably 10 to 25.

M, Mₙ (n being an integer): Metals (either charged or uncharged), whereby two Metals Mₙ and Mₘ are independently selected from each other unless otherwise indicated.

According to a preferred embodiment of the present invention, the substrate material includes a material with comprises an alkene group.

In the sense of the present invention, the term “a material with comprises an alkene group” means that the following functional group

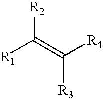

is present somewhere in the chemical structure of the molecule. R₁ and R₂ may be independently selected from each other and may be identical or different.

Preferably, R₁, R₂, R₃ and/or R₄ are independently selected out of a group comprising hydrogen, hydroxyl, halogen, formyl, carboxy- and/or carbonyl derivatives, alkyl, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine.

In case that the substrate material includes a material with comprises an alkene group, a phase transfer may be reached by photochemically isomerization of the alkene group.

Usually, an alkene group exists in two conformations, cis and trans

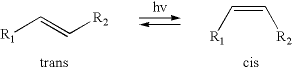

It is possible to isomerize between the two isomers photochemically. In some applications, the isomerization may also be conducted thermally although this less prominent than in the case of the presence of an azo group.

Due to the different structure of the two isomers which have different topical as well as chemical properties, a shift in the alkene-group containing material can—often quite similarly as for the azo-group described above—be used to induce a phase transition in the substrate material. This goes especially for an insofar preferred embodiment (which will be described in great detail later on) in which the substrate material also contains a liquid crystal material.

According to a preferred embodiment of the present invention, the substrate material includes a material with comprises an alkene group in that that one or more of the following materials and/or possibilities are present:
According to a preferred embodiment of the present invention, the substrate material includes a material which comprises an alkene group and which is also capable to influence and/or cause a flow of fluid or parts thereof in and/or with the sample by undergoing phase transition(s).
According to a preferred embodiment of the present invention, the substrate material includes a material which comprises an alkene group and which is—preferably upon isomerization—capable of influencing and/or causing a phase transition in or with a second material, which then influences and/or causes a flow of fluid or parts thereof in and/or with the sample.

According to a preferred embodiment of the present invention, the substrate material includes a material with comprises an alkene group in that that one or more of the following materials and/or possibilities are present:
A material comprising an alkene group and a further group capable of undergoing a phase transition in the same molecule; preferably, the material is a liquid crystal material;
A material comprising an alkene group which is added to a second material capable of undergoing a phase transition in the same molecule; preferably, this second material is a liquid crystal material;
A polymeric material where alkene groups are present in side-chain moieties;
A polymeric liquid crystal material which also comprises alkene groups; here the polymeric material may be a homopolymer and/or a copolymer.

According to a preferred embodiment of the present invention, the substrate material includes a material which comprises an alkene group and which shows liquid crystal properties when the alkene group is in one conformation and which shows different liquid crystal properties when the alkene group is in the other conformation.

The term “different liquid crystal properties” includes one or more of the following:
the material has no or only very little liquid crystal properties when the alkene group is in the other conformation;
the material still shows liquid crystal properties when the alkene group is in the other conformation, but the phase transition points and/or conditions are different.

According to a preferred embodiment of the present invention, the substrate material includes a material with comprises at least one group with the structure II:

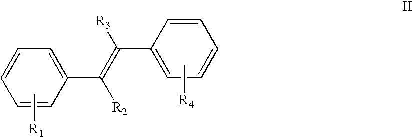

wherein R1, R2, R3 and/or R4 are independently selected out of a group comprising hydrogen, hydroxyl, halogen, formyl, carboxy- and/or carbonyl derivatives, alkyl, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine.

According to a preferred embodiment of the present invention, the substrate material includes a material with comprises at least one group with the structure III:

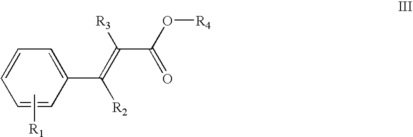

wherein R1, R2, R3 and/or R4 are independently selected out of a group comprising hydrogen, hydroxyl, halogen, formyl, carboxy- and/or carbonyl derivatives, alkyl, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine.

According to a preferred embodiment of the present invention, the substrate material includes a material with comprises an imino group.

In the sense of the present invention, the term “a material with comprises an imino group” means that the following functional group

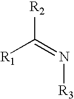

is present somewhere in the chemical structure of the molecule. R₁ and R₂ may be independently selected from each other and may be identical or different.

Preferably, R₁, R₂ and/or R₃ are independently selected out of a group comprising hydrogen, hydroxyl, halogen, formyl, carboxy- and/or carbonyl derivatives, alkyl, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine.

In case that the substrate material includes a material with comprises an imino group, a phase transfer may be reached by photochemically isomerization of the imino group.

Usually, an imino group exists in two conformations, cis and trans

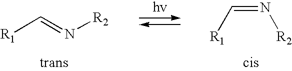

It is possible to isomerize between the two isomers photochemically. In some applications, the isomerization may also be conducted thermally although less prominent than in the case of the presence of an azo group.

Due to the different structure of the two isomers which have different isotropical as well as chemical properties, a shift in the imino-group containing material can—often quite similarly as for the azo-group described above—be used to induce a phase transition in the substrate material. This goes especially for an insofar preferred embodiment (which will be described in great detail later on) in which the substrate material also contains a liquid crystal material.

According to a preferred embodiment of the present invention, the substrate material includes a material with comprises an imino group in that that one or more of the following materials and/or possibilities are present:
According to a preferred embodiment of the present invention, the substrate material includes a material which comprises an imino group and which is also capable to influence and/or cause a flow of fluid or parts thereof in and/or with the sample by undergoing phase transition(s).
According to a preferred embodiment of the present invention, the substrate material includes a material which comprises an imino group and which is—preferably upon isomerization—capable of influencing and/or causing a phase transition in or with a second material, which then influences and/or causes a flow of fluid or parts thereof in and/or with the sample.

According to a preferred embodiment of the present invention, the substrate material includes a material with comprises an imino group in that that one or more of the following materials and/or possibilities are present:
A material comprising an imino group and a further group capable of undergoing a phase transition in the same molecule; preferably, the material is a liquid crystal material;
A material comprising an imino group which is added to a second material capable of undergoing a phase transition in the same molecule; preferably, this second material is a liquid crystal material;
A polymeric material where imino groups are present in side-chain moieties;
A polymeric liquid crystal material which also comprises imino groups; here the polymeric material may be a homopolymer and/or a copolymer.

According to a preferred embodiment of the present invention, the substrate material includes a material which comprises an alkene group and which shows liquid crystal properties when the alkene group is in one conformation and which shows different liquid crystal properties when the alkene group is in the other conformation.

According to a preferred embodiment of the present invention, the phase substrate material includes a liquid crystal material. By using such a material, the phase-transition between the smectic, nematic and/or isotropic state may be used to control the flow of the fluid.

In some applications of the present invention, in the case of the liquid crystalline transition often the transition is thermo-reversible, i.e. upon heating and upon cooling the transition occurs at about the same temperature. In the case of the crystalline transition melting upon heating usually occurs at a higher temperature than crystallization during cooling. This is because the nucleation of the crystallization retards the crystallization process and the phenomenon is known as supercooling where an isotropic liquid remains for a while in its non-equilibrium thermodynamic state. Improvement of the thermo reversibility can be enforced by the addition of nucleation agents.

A liquid crystal material in the sense of the present invention means especially an organic liquid material whose physical properties resemble those of a crystal in the formation of loosely ordered molecular arrays similar to a regular crystalline lattice and the anisotropic refraction of light. Different degrees of order are possible. The less ordered liquid crystal state is the nematic state. Here all molecules on the average are oriented into a similar direction, but there is no order in their centers of gravity. Higher ordered state are given by the so-called smectic phases in which the molecules on the average have a directional order and a positional order. Typically the molecules are ordered within layers. Depending on the degree of organization of the molecules within the layers one recognized a distinction in smectic phases denoted by a capital letter. For instance in the smectic A phase the molecules are ordered in layers with their average orientation perpendicular to the layer surface. Within the layer there is no positional order. In the smectic B phase the molecules are positioned hexagonally in the layers. The smectic C state resembles the state of order of smectic A, but the molecules are aligned under an angle with the layer surface. In the smectic D state the molecules are packed on a cubic lattice, etc. This all is common knowledge for those who are skilled in the field. In the case of polymeric liquid crystals the material exhibit the same types of order in their molecular parts, but the viscosity has become much higher such that in the liquid crystalline state they behave like pastes or elastomers in the case of crosslinked polymer systems.

By using a liquid crystal material, a control of the fluid in and/or with the substrate material can be achieved easily and effectfully. Without being determined, it is believed that one of the following mechanisms is responsible at least to a great deal to this effective control:

Liquid crystals tend to expel ‘foreign’ species driven by strong intermolecular interactions and the related elastic constants of the liquid crystal. If for instance foreign molecules (e.g. a fluid containing particles) are added to a liquid crystal system that is e.g. heated to a temperature where it is in its isotropic state, the particles distribute homogeneously. As soon as the temperature is lowered such that the material undergoes its phase transition e.g. to the nematic liquid crystalline state, the suspended particles are driven to concentrate themselves in the still isotropic areas.

It should be noted that the above described mechanism describes a phase transition from the isotropic state to the nematic state. It goes without saying that also further phase transitions, which can be effected in liquid crystal materials, e.g. from the smectic state to the nematic state may be employed as well.

According to a preferred embodiment of the present invention, the substrate material includes an aligned liquid crystal material. An Alignment of the liquid crystals has shown for some applications to be beneficial to obtain more control over the directional flow, especially in case the detection of the analytes is optical based.

The term “alignment” in the sense of the present invention means especially that the liquid crystals exhibit long range orientation order. On average the long axis of the liquid crystalline molecules are oriented approximately parallel in a preferred direction. Optionally, the liquid crystals exhibit translation order as well.

According to a preferred embodiment of the present invention, the substrate material comprises a material according to structure IV:

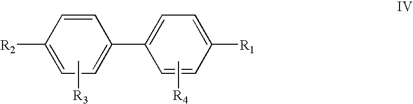

wherein R1, R2, R3 and/or R4 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

According to a preferred embodiment of the present invention, the substrate material comprises a material according to structure V:

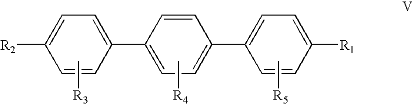

wherein R1, R2, R3, R4 and/or R5 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

According to a preferred embodiment of the present invention, the substrate material comprises a material according to structure VI:

R₁—R₃—R₂ VI

wherein R1, and/or R2 are independently selected out of a group comprising cycloalkyl, aryl, arylene, halogenaryl, heteroaryl, heteroarylene, heterocycloalkylene, heterocycloalkyl, halogenheteroaryl, ketoaryl, halogenketoaryl, ketoheteroaryl either unsubstituted or substituted with one or more substituents selected out of the group comprising hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether;

and R3 is chosen out of the group comprising carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, azo, azoxy, imino, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

According to a preferred embodiment of the present invention, R3 is chosen out of the group comprising ethylene, ethyl, alkinyl, ester, thioester, azo, azoxy, imino, butyl, 2-butylen, cyclohexyl, 2-cyclohexylen.

According to a preferred embodiment of the present invention, the substrate material comprises a material chosen from the group comprising the structures VII to X:

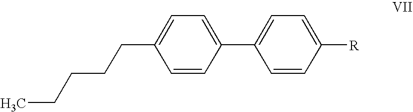

whereby R is chosen out of the group comprising halogens and pseudohalogens

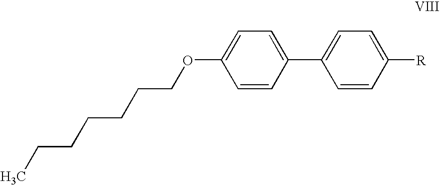

whereby R is chosen out of the group comprising halogens and pseudohalogens

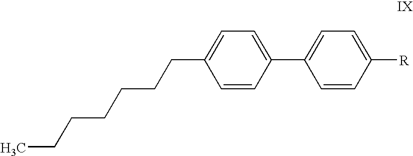

whereby R is chosen out of the group comprising halogens and pseudohalogens

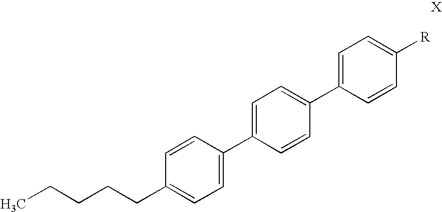

whereby R is chosen out of the group comprising halogens and pseudohalogens

or mixtures thereof. Preferably, the substrate material comprises a mixture of the compounds VII to X.

A preferred mixture of these materials is commercially available under the name of E7 (Merck KGaA, Frankfurter Str. 250, D-64293 Darmstadt, Germany), with all groups R being R=—CN. It is nematic at room temperature and has its nematic to isotropic transition at 58° C. Manipulating its phase transition can easily be done by blending it with other materials. For instance the material that is denoted as structure III has a nematic to isotropic transition of 35.5° C. Just increasing the amount of this compound decreases the transition almost linearly. If on the other hand higher temperatures are needed the component III should be added in a higher quantity.

This blend of so-called cyanobiphenyls is suited for analytes with a medium polarity. In case the analyte consist of molecules of low polarity, the group R is preferably chosen to be halogen, more preferably a fluoro group.

According to a preferred embodiment of the present invention, the substrate comprises a material selected out of the group comprising the structures VII to X and mixtures thereof together with the following compound XI

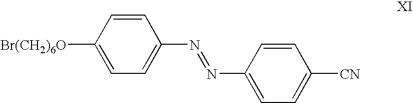

Preferably, the substrate material comprises a mixture of the compounds with the structures VII to X and is admixed with the compound XI in a quantity of ≧15 to ≦25 wt %.

According to a preferred embodiment of the present invention, the substrate material comprises a material according to structure XII to XVII:

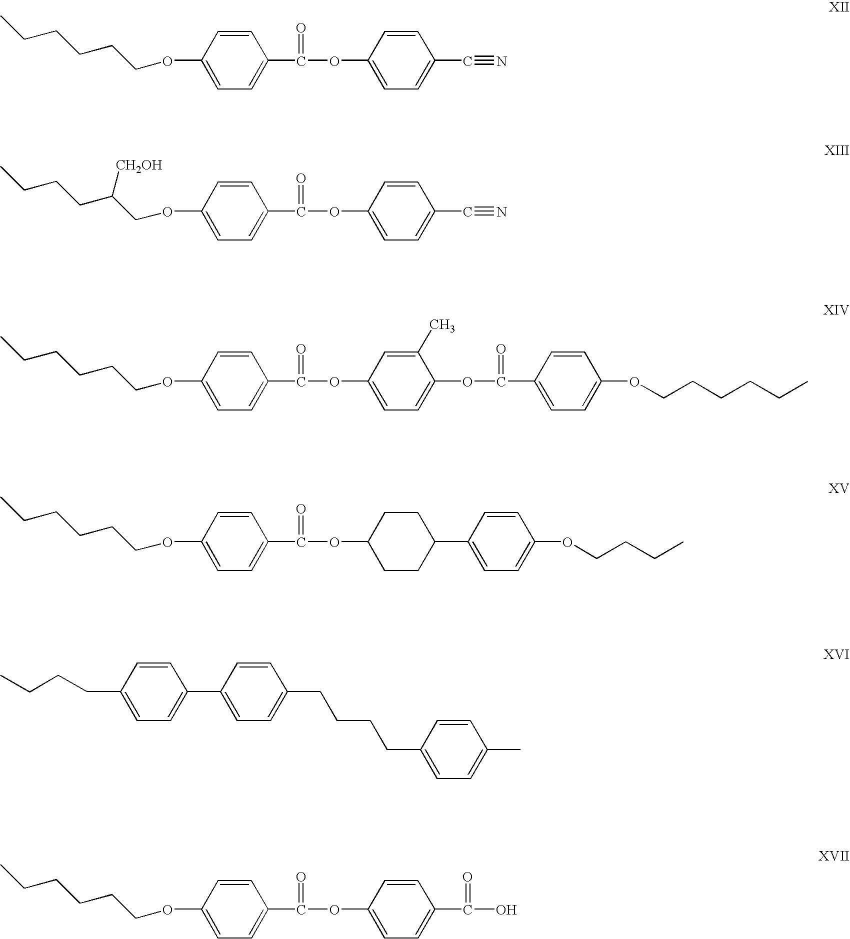

or mixtures thereof.

According to a preferred embodiment of the present invention, the substrate material comprises a polymeric liquid crystal material. These materials have shown to be suitable within the present invention.

According to a preferred embodiment of the present invention, the substrate material comprises a polymeric material selected out of the group polyacrylate, a polymethacrylate, a polyether, a polyester, a polypeptide or a polysiloxane or mixtures thereof, whereby liquid crystal molecules and/or structural moieties are attached as side groups to the polymer main chain.

According to a preferred embodiment of the present invention, the substrate material comprises a polymeric material selected out of the group polyacrylate, a polymethacrylate, a polyether, a polyester, a polypeptide or a polysiloxane or mixtures thereof, whereby photoisomerizable molecules and/or structural moieties are attached as side groups to the polymer main chain. Preferably, these photoisomerizable molecules and/or structural moieties are selected out of the group comprising azo compounds, alkenes and mixtures thereof.

According to a preferred embodiment of the present invention, the substrate material is a liquid crystal material, whereby the liquid crystal side chain moieties comprise at least one of the following structures XVIII, XIX and XX

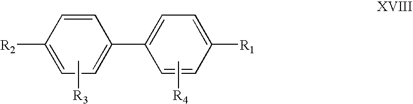

wherein R1, R2, R3 and/or R4 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

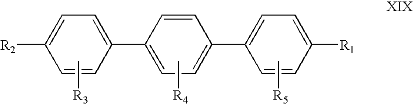

wherein R1, R2, R3, R4 and/or R5 are independently selected out of a group comprising hydrogen, hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether.

R₁—R₃—R₂ XX

wherein R1, and/or R2 are independently selected out of a group comprising cycloalkyl, aryl, arylene, halogenaryl, heteroaryl, heteroarylene, heterocycloalkylene, heterocycloalkyl, halogenheteroaryl, ketoaryl, halogenketoaryl, ketoheteroaryl either unsubstituted or substituted with one or more substituents selected out of the group comprising hydroxyl, halogen, pseudohalogen, formyl, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether;

and R3 is chosen out of the group comprising carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, azo, azoxy, imino, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether; preferably, R3 is chosen out of the group comprising ethylene, ethyl, alkinyl, ester, thioester, azo, azoxy, imino, butyl, 2-butylen, cyclohexyl, 2-cyclohexylen.

The term “whereby the liquid crystal side chain moieties comprise the following structures” means especially that the structural moieties are present somewhere in the liquid crystal material.

According to a preferred embodiment, the percentage of side chains of the polymeric liquid crystal material, on which liquid crystal molecules and/or structural moieties are attached to is ≧0 and ≦100%, preferably ≧5 and ≦100%, more preferably ≧20 and ≦100% and most preferred ≧50 and ≦100%.

According to a preferred embodiment, the substrate material comprises a liquid crystal material is a partial-crosslinked polyether material comprising the following structural moieties XXI, XXII, and XXIII,

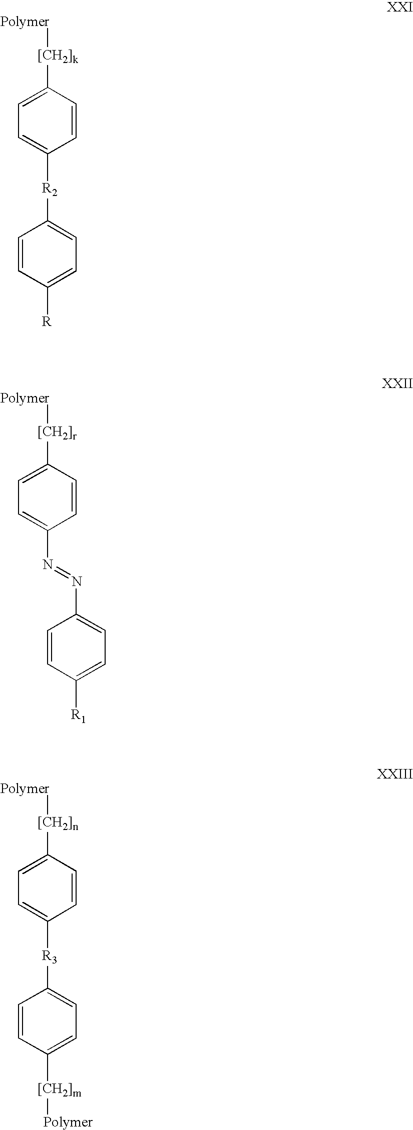

whereby R and R1 are independently selected from the group comprising halogens and pseudohalogens; R2 and R3 are independently selected from the group comprising single bond, carboxy- and/or carbonyl derivatives, alkyl, long-chain alkyl, alkoxy, long-chain alkoxy, cycloalkyl, halogenalkyl, aryl, arylene, halogenaryl, heteroaryl, heteroarylene, azo, azoxy, imino, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine, polyether; and

k, r, n and m are independently chosen from each other integers from ≧2 to ≦18, preferably ≧3 to ≦12, more preferred ≧4 to ≦8.

According to a preferred embodiment of the present invention, the substrate material comprises a liquid crystal material is a partial-crosslinked polyether material comprising the following structural moieties XXI, XXII and XXIII as described, whereby the ratio of moieties according to structure XXI and XXII to moieties according to structure XXIII is from ≧2:1 to ≦2000:1, preferably ≧4:1 to ≦1000:1, more preferred ≧10:1 to ≦500:1.

According to a preferred embodiment of the present invention, the substrate material comprises a liquid crystal material is a partial-crosslinked polyether material comprising the following structural moieties XXI, XXII and XXIII as described, whereby the ratio of moieties according to structure XXI to moieties according to structure XXII is from ≧0.2:1 to ≦20:1, preferably ≧0.4:1 to ≦10:1, more preferred ≧1:1 to ≦4:1.

The object of the present invention is furthermore solved by a method of influencing the flow of a fluid sample in or with a substrate material according to the present invention, whereby the method comprises the step of causing a phase transition in at least on desired area of the substrate material.

The object of the present invention is furthermore solved by a method of influencing the flow of a fluid sample in or with a substrate material according to the present invention, whereby the method comprises the step of causing at least one phase transition in at least on desired area of the substrate material.

The object of the present invention is furthermore solved by a device comprising a substrate material as described above, whereby the device is equipped with lighting means in at least one desired area of the substrate material.

According to a preferred embodiment of the present invention, the illumination means include a laser means.

According to a preferred embodiment of the present invention the illumination means include a diode light source.

According to a preferred embodiment of the present invention the illumination means include an array of diodes which by switching can subsequently illuminate adjacent areas and thereby transport the analyte over the surface.

A substrate material, a method and/or device according to the present invention may be of use in a broad variety of systems and/or applications, amongst them one or more of the following:
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
nano- and micro-fluidic devices
fluid pumping devices
drug release and drug delivery systems (in particular transdermal and implantable drug delivery devices).

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

Additional details, features, characteristics and advantages of the object of the invention are disclosed in the dependent claims, the figures and the following description of the respective figures and examples, which—in an exemplary fashion—show several preferred embodiments of a substrate material as well as a device according to the invention.

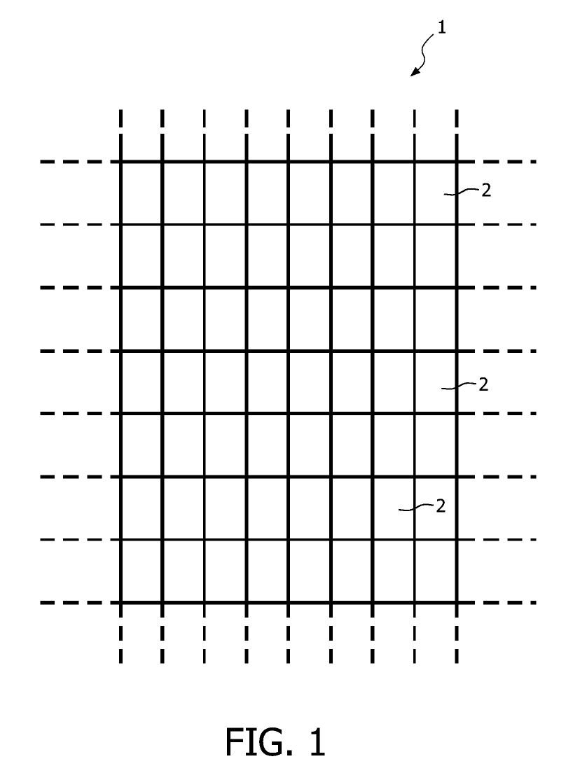

FIG. 1 shows a very schematic cross-sectional cut-out view of a substrate material according to a first embodiment of the present invention;

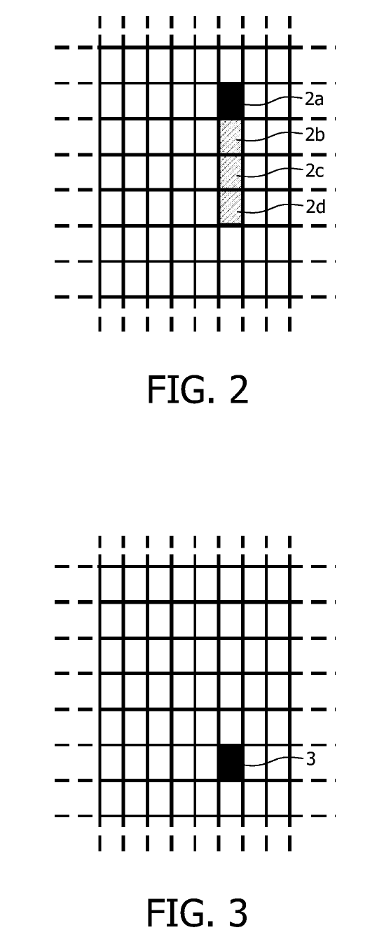

FIG. 2 shows the substrate material of FIG. 1 after addition of a sample and prior to the inducement of phase transition in certain selected areas in the substrate material;

FIG. 3 shows the substrate material of FIG. 2 after several phase transition in selected areas in the substrate material;

FIG. 1 shows a very schematic cross-sectional cut-out view of a substrate material according to a first embodiment of the present invention. The substrate material 1 consists out of a plurality of cells 2, which are usually somewhat square or rectangular in shape. By means, e.g. lighting means as will described later on, a phase transition can be induced in each of the cells 2 essentially separately or independently.

FIG. 2 shows the substrate material of FIG. 1 after addition of a sample and prior to the inducement of phase transition in certain selected areas in the substrate material. In this purely exemplarily embodiment, the sample has been added to cell 2a. After the addition and an optional drying step phase transitions are conducted in cells 2b, 2c and 2d, thus causing the sample or parts thereof to flow from cell 2a to 2d. It should be noted, that according to the chosen application, only parts of the sample e.g. macroparticles may be caused to flow within the substrate material 1 whereas the rest of the sample will remain in cell 2a.

According to an alternative and also preferred embodiment of the present invention, the substrate material is adapted in that way that a size-selective or size-dependent flow of macroparticles in the fluid in and/or with the substrate material is influenced and/or caused by phase transitions in selected areas of the sample. In FIG. 2 this would mean that smaller particles would e.g. be caused to flow to cell 2d, whereas larger particles would e.g. be caused to flow to cell 2b and medium-sized particles to cell 2c.

FIG. 3 shows the substrate material of FIG. 2 after several phase transition in selected areas in the substrate material. The fluid sample (or parts thereof as described below) will then concentrate in a cell of the substrate material different from the cell where the sample was originally added to, e.g. cell 3. By further phase transition to selected areas of the substrate material, the fluid sample (or parts thereof) may be shifted all over the substrate material 1 as desired.

It should be noted that (although this is not shown in the figs.) the cells are usually equipped with binding substances selective for certain analytes in the sample or the device, in which the substrate material is located in, is provided with further means which contain these binding substances (e.g. in the form that a further layer of material is provided, which contains such binding substances). When the sample comes across these binding substances, the corresponding analytes will be bound to the binding substances for analysis. It is obvious that by the possibility to “move” or “shift” the sample around the substrate material, the selectivity and resolution of the analysis device is greatly enhanced and the minimal required amount of fluid is decreased to a great extend as compared with the prior art.

The inducement of phase transitions by light through the lighting means may be achieved by a plurality of means, including one or more of the following
The induction may be done by a laser means. By directing the laser to different cells, the flow of the fluid sample or parts thereof in and/or with the sample may be controlled easily and effectfully.
By using a laser means in some applications also the sample might heat up in which case the underlying mechanism for the phase transition is a combination of light and heat. Possibly the heating part can be enhanced by the addition of dye molecules to the liquid crystal medium to increase light absorption at the wavelength that the laser system emits.
The induction may also be done by lighting the substrate material with e.g. a UV-lamp and covering the parts of the substrate material with a mask, where no phase transition is to occur. By changing the masks, also a controlled movement of the fluid sample or parts thereof in and/or with the sample is achieved and controlled easily and effectfully. The mask pattern-technique is known e.g. from the mask-pattern technique where photolabile protective groups are removed when building up DNA-libraries on a chip (Affymetrix)
According to another preferred embodiment, lighting means such as UV diodes are implemented in the analytical system, e.g. at the edges of a micro capillary. In the exposed areas the analyte will concentrate itself.
According to another preferred embodiment the light source is coupled to an electro-optical light switch to adjust the position of the illuminated areas. The electro-optical light switches are for instance known from projection display systems where the beam can be modulated either by a liquid crystal cell provided with a polarizing system or by a system based on an array of switchable mirrors known in the field as a digital mirror device or deformable mirror device (Texas Instruments Incorporated, 12500 TI Boulevard Dallas, Tex. 75243-4136, USA). Both with liquid crystal beam controller and with the digital (deformable) mirror device the light beam is controlled on a pixel level and can be easily connected to a computer such that the light intensity on the substrate material can be controlled by simple graphic software programs.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. A substrate material for analyzing a sample for at least one analyte, whereby a lighting means is arranged for causing photo-inducible phase transitions of a liquid crystal in the sample exerting a force to the sample thereby causing a flow of the sample.

2. A substrate material according to claim 1, whereby the phase transitions in the substrate material include reversible phase transitions.

3. A substrate material according to claim 1, whereby the substrate material includes a material which comprises an azo group, an imino group and/or an alkene group.

4. A substrate material according to claim 1, whereby the substrate material includes a material which comprises at least one group with the structure I 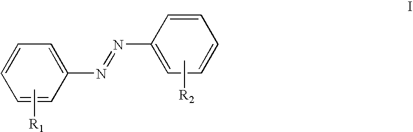
wherein R1 and/or R2 are independently selected out of a group comprising hydrogen, hydroxyl, halogen, formyl, carboxy- and/or carbonyl derivatives, alkyl, cycloalkyl, halogenalkyl, aryl, arylene, halogenaryl, heteroaryl, heteroarylene, heterocycloalkylene, heterocycloalkyl, halogenheteroaryl, alkenyl, halogenalkenyl, alkinyl, halogenalkinyl, keto, ketoaryl, halogenketoaryl, ketoheteroaryl, ketoalkyl, halogenketoalkyl, ketoalkenyl, halogenketoalkenyl, phosphoalkyl, phosphonate, phosphate, phosphine, phosphine oxide, phosphoryl, phosphoaryl, sulphonyl, sulphoalkyl, sulphoarenyl, sulphonate, sulphate, sulphone, amine.

5. A method of influencing the flow of a sample at a substrate material, whereby the flow of the sample is caused by a photoinduced phase transition of liquid crystals.

6. A device comprising a substrate material according to claim 1, whereby the device is equipped with lighting means at least one desired area of the substrate material.

7. A device according to claim 6, whereby the lighting means include a laser means, a diode light source and/or an array of diodes.

8. A system incorporating a substrate material for analyzing a sample for at least one analyte, whereby a lighting means is arranged for causing photo-inducible phase transitions of a liquid crystal in the sample exerting a force to the sample thereby causing a flow of the sample, adapted to conduct the method of claim 5 and being used in one or more of the following applications:
biosensors used for molecular diagnostics
rapid and sensitive detection of proteins and nucleic acids in complex biological mixtures such as e.g. blood or saliva
high throughput screening devices for chemistry, pharmaceuticals or molecular biology
testing devices e.g. for DNA or proteins e.g. in criminology, for on-site testing (in a hospital), for diagnostics in centralized laboratories or in scientific research
tools for DNA or protein diagnostics for cardiology, infectious disease and oncology, food, and environmental diagnostics
tools for combinatorial chemistry
analysis devices
nano- and micro-fluidic devices
fluid pumping devices
drug release and drug delivery systems (in particular transdermal and implantable drug delivery devices).
